(19) United States
(12) Patent Application Publication
(10) Pub. No.: US 2024/0065939 A1
(43) Pub. Date: Feb. 29, 2024

(54) SENSOR APPARATUS WITH AN ACCELERATION SENSOR FOR MONITORING MOVEMENT AND TRANSMITTING DATA ON A MEDICAMENT CONTAINER, MONITORING SYSTEM AND MONITORING METHOD

(71) Applicant: IPB INTELLIGENT PILL BOX UG (HAFTUNGSBESCHRÄNKT), Berlin (DE)

(72) Inventors: Georg HACHMANN, Berlin (DE); Miriam AGUILERA, Woodland Hills, CA (US)

(73) Assignee: IPB INTELLIGENT PILL BOX UG (HAFTUNGSBESCHRÄNKT), Berlin (DE)

(21) Appl. No.: 18/271,216

(22) PCT Filed: Jan. 6, 2022
(86) PCT No.: PCT/EP2022/050217

§ 371 Date: Jul. 6, 2023

(30) Foreign Application Priority Data
Jan. 6, 2021 (DE) 20 2021 100 044.9
Aug. 13, 2021 (DE) 10 2021 208 918.6
Aug. 13, 2021 (DE) 10 2021 208 919.4

(51) Int. Cl. A61J 1/18; G08B 21/18; G08B 27/00

(52) U.S. Cl. CPC A61J 1/18; G08B 21/182; G08B 27/005; A61J 2200/70

(57) ABSTRACT

It is provided a sensor apparatus for attachment to a medicament container, comprising at least one acceleration sensor via which a movement of the medicament container can be detected when the sensor apparatus is attached to the medicament container, and an electronic connection device which is configured to wirelessly transmit data from the sensor apparatus to the computer unit.

### CROSS-REFERENCE TO A RELATED APPLICATION

This application is a National Phase patent application of International Patent Application Number PCT/EP 2022/050217, filed on Jan. 6, 2022, which claims priority of German Utility Model Application Number 20 2021 100 044.9, filed on Jan. 6, 2021, of German Patent Application Number 10 2021 208 918.6, filed on Aug. 13, 2021 and of German Patent Application Number 10 2021 208 919.4, filed on Aug. 13, 2021.

### BACKGROUND

The proposed solution relates in particular to a sensor apparatus for attachment to an object in the form of a medicament container, such as for example a medicament box, a medicament bottle, a pill blister or a medicament package, in particular a pill box, a pill bottle, a pill carton, or a medicament bottle for a liquid or powdered medicament.

A proposed sensor apparatus comprises at least one acceleration sensor via which a movement of the object can detected when the sensor apparatus is attached to the object. Furthermore, the sensor apparatus is capable of wirelessly transmitting data on a detected movement to a computer unit via an interface. Via an electronic connection device, the sensor apparatus can be coupled to the computer unit for a subsequent wireless transmission of data. In this connection it is known, for example, that a sensor apparatus which can communicate with a computer unit via Bluetooth initially carries out a pairing process with the computer unit.

In sensor apparatuses known so far from practice, which for example can be attached to an object in order to detect and possibly log a movement of an object and/or trigger an alarm in dependence on a particular movement, a wireless coupling to the computer unit before or after the attachment of the sensor apparatus to the object regularly can only be realized by actuation of an operating element to be separately provided on the sensor apparatus, for example in the form of a switch or button. As a corresponding operating element then must still be accessible for example on the sensor apparatus for coupling, a coupling operation possibly can be triggered only before attachment to the object, in particular when the sensor apparatus should no longer be accessible after the attachment. In a sensor apparatus attached to the object in a hidden manner, coupling to the computer unit therefore no longer is possible after its attachment to the object. Moreover, an operating element to be additionally provided on the sensor apparatus increases the costs and the size of the sensor apparatus.

Furthermore, previously used sensor apparatuses frequently involve the problem of a comparatively large energy demand. When corresponding sensor apparatuses for example are attached to an object in order to monitor its position and detect an abusive displacement of the object, such sensor apparatuses so far have regularly been configured in such a way that they every detected movement will be wirelessly communicated to the computer unit via the interface. In this way, a possible energy accumulator of the sensor apparatus, via which the electronics are supplied with electrical current, is used up relatively quickly.

Furthermore, especially in medicament containers and monitoring systems including at least one medicament container, it is widely known to monitor the intake of a medicament stored in a medicament container, in order to avoid that a predetermined time for taking the medicament is missed, in particular when an elderly person is responsible for the intake and no nursing staff or family members monitor the intake.

For this reason, it is already known to use electronic monitoring devices for monitoring purposes, which acoustically and/or visually signal to a user when the time for taking a medicament has come. Such monitoring devices then for example are also capable of triggering an alarm when the taking of a medicament has been omitted. Typically such monitoring devices are complex and hence comparatively costly. Therefore, individual providers combine the use of a corresponding electronic monitoring device with a subscription model in order to save a user the comparatively high acquisition costs for the monitoring system, but at the same time link its use to a subscription that incurs quite considerable monthly costs.

### SUMMARY

Against this background, it is the object underlying the proposed solution to provide an improved sensor apparatus and to provide a simpler and less expensive possibility of electronically monitoring the intake of medicaments by means of a medicament box in which the medicament is held.

This object is achieved with a sensor apparatus, a method and a monitoring system as described herein.

A proposed sensor apparatus for example can comprise an electronic connection device that is configured to
in response to a sequence of at least two movements detected via the acceleration sensor and fulfilling at least one predefined detection criterion within a predetermined time period a) trigger a transmission of data from the sensor apparatus to the computer unit and/or b) trigger coupling of the sensor apparatus to the computer unit, and/or
generate an alarm signal, when a removal signal representative of the removal of a medicament from the medicament container has not been generated in a predefined time window via the at least one acceleration sensor.

A proposed sensor apparatus according to one aspect thus for example comprises an electronic connection device which is configured, in response to a sequence of at least two movements detected via an acceleration sensor of the sensor apparatus and fulfilling at least one predefined detection criterion, to trigger a transmission of data from the sensor apparatus to the computer unit and/or to trigger (wireless) coupling of the sensor apparatus to a computer unit within a predetermined time period, to which computer unit data on at least one detected movement of the sensor apparatus are to be subsequently transmitted.

Coupling of the sensor apparatus to the computer unit for the subsequent wireless transmission of data here in particular refers to a first-time establishment of a data connection for a future data transmission so that data can be transmitted to the computer unit and can be properly received from the computer unit and can be further processed. Thus, coupling for example refers to so-called pairing in the case of a communication via Bluetooth. In accordance with the proposed solution it now is provided to trigger such coupling in response to the movements detected by means of the acceleration sensor. To be able to subsequently transmit data on one or more detected movements to the computer unit without coupling again, an operating event thus is triggered (possibly solely) by using the at least one acceleration sensor. Such an operating event, which leads to wireless coupling of the sensor apparatus to the computer unit, hence can be realized independently of the actuation of a separate operating element on the sensor apparatus or on an electronic control unit or control software/control application cooperating with the sensor apparatus.

Coupling of the sensor apparatus to the computer unit thus can be triggered for example solely in dependence on whether within a predetermined time window or time interval, for example calculated from a first detected movement until expiration of a timer of the sensor apparatus, two particular movements are identified via at least one acceleration sensor of the sensor apparatus. A particular sequence of movements of the sensor apparatus thus triggers a coupling operation at the sensor apparatus. In this way, a coupling operation can be triggered comparatively easily before or also even after the attachment of the sensor apparatus to an object to be monitored, which in the present case is formed by a medicament container. After a successfully performed coupling operation, a data connection is established between the sensor apparatus and the computer unit in order to be able to transmit data on at least one detected movement to the computer unit, which data can then be evaluated at the computer unit or a system connected thereto.

An acceleration sensor of the sensor apparatus for example can form part of a an inertial sensor comprising the acceleration sensor and/or a gyroscope. Possible (event) data on at least one detected movement, which in particular result from signals of the at least one acceleration sensor of the sensor apparatus, furthermore can comprise for example a starting time and/or end time and/or a duration of a detected movement, a type (for example rotation or translation) and/or direction of a movement, position data on at least one position reached with the detected movement (absolutely in space or relative to a previously taken position) and/or a (global) date and/or time indication as to when the detected movement has occurred.

From such data on at least one detected movement it can thus be determined for example whether the medicament container to which the sensor apparatus is attached has been moved properly and in particular at a time intended therefor. A medicament container designed as a medicament box here for example includes at least one compartment for storing at least one medicament and is to be handled manually by a user in order to remove the medicament from the compartment for taking. Analogously, other medicament containers such as for example a medicament bottle, a pill blister or a medicament package also are to be handled manually by a user in order to remove a medicament therefrom.

For example, the connection device of the sensor apparatus is configured to trigger the coupling of the sensor apparatus to the computer unit when a sequence of at least two successive movements has been detected, wherein during each of the successive movements an acceleration of the sensor apparatus occurs, which exceeds an acceleration setpoint value stored as a detection criterion. When it is now detected for example by means of the at least one acceleration sensor of the sensor apparatus that the sensor apparatus is moved in a particular way at least two times within a predetermined and for example comparatively short time window, the coupling of the sensor apparatus to the computer unit is automatically triggered. For example, the sensor apparatus can abruptly be moved two times in the same spatial direction within a defined time period of 15 seconds, in particular of 10 seconds, and in particular for example of 5 seconds, in order to thereby trigger an operating event for coupling the sensor apparatus to the computer unit. In this connection it can be provided for example that the connection device triggers the wireless coupling of the sensor apparatus to a computer unit, when a user manually carries out at least two striking or tapping movements within a time window, for example a short time window of for instance 15 or 10 or 5 seconds, as defined above, by means of the sensor apparatus. In the present case, a tapping movement also is understood to be a movement for a so-called “double tap”.

In a possible development, the at least two successive movements, for triggering a coupling operation at the sensor apparatus, must each comprise an acceleration of the sensor apparatus from an idle state and must be effected along a matching spatial direction. An acceleration out of an idle state here in particular comprises the acceleration out of a stationary or approximately stationary state and hence a state in which a speed of a movement of the sensor apparatus lies below a speed threshold value and for example is equal to 0. Two successive movements which fulfill these conditions for example would be the striking or tapping movements as explained above, in which the sensor apparatus (before attachment to the medicament container or together with the medicament container to which the sensor apparatus is attached already) is accelerated along a spatial direction, subsequently moved back in the opposite direction and then accelerated again along the spatial direction. At least two successive movements with an acceleration out of an idle state and along a matching spatial direction thus lead to the identification as an operating event and the triggering of a coupling operation.

In one design variant, the connection device is configured, in response to a sequence of two or more movements of the sensor apparatus, which are detected and predefined via the acceleration sensor, to trigger a function that is different from the coupling of the sensor apparatus to the computer unit. In such a design variant, at least one further function of the sensor apparatus consequently can be actuated via the acceleration sensor. This further function for example can be triggerable only when previously a successful coupling of the sensor apparatus to the computer unit has taken place. Only then can the other function be triggered in response to the (further) sequence of the two or more predefined movements of the sensor apparatus.

In principle, for example, the time period in which the at least two movements for triggering the coupling of the sensor apparatus to the computer unit must be effected can be different from a time period in which the two or more predefined movements of the sensor apparatus must occur in order to trigger the other function. When for example for a (first-time) coupling of the sensor apparatus to the computer unit two particular movements within a first time window are sufficient, two or more movements must be carried out with the sensor apparatus within a second, shorter time window for triggering a further function.

Alternatively or additionally, the number of the at least two successive movements, which for triggering the coupling of the sensor apparatus to the computer unit must each fulfill a predefined detection criterion, can be different from a number of two or more successive movements which must occur in order to trigger the other function. Correspondingly, the number of movements of the sensor apparatus, which are to be carried out successively in a predetermined time window, can vary in dependence on whether the coupling of the sensor apparatus to the computer unit or the other function is to be triggered. In addition, the size of the time windows can also be different. In one exemplary embodiment, for example, the sensor apparatus is adapted to trigger a coupling operation in the case of two successive striking or tapping movements within 10 or 5 seconds, or in the case of a one-time “double tap”. A sequence of four striking or tapping movements carried out one after the other with the sensor apparatus within a time window identical in size or different in size of for example 10 or 5 seconds, or a two-time “double tap”, in turn are assessed as a valid operating event for triggering the other function.

For example, the other function comprises a wireless transmission of data on at least one of the detected movements of the sensor apparatus (via the interface of the sensor apparatus) to the computer unit. In this way, a user can specifically trigger a wireless transmission of (previously collected and/or stored) data on at least one detected movement of the sensor apparatus to the computer unit by carrying out targeted movements with the sensor apparatus. It thereby is achieved in particular that data on one or more detected movements are transmitted to the computer unit exclusively or at least additionally as necessary and only via a targeted intervention on the part of a user (which leads to a movement of the sensor apparatus in a particular way). The energy demand of the sensor apparatus thereby can possibly be reduced considerably, as the sensor apparatus transmits at least some data on at least one detected movement to the computer unit only when needed and not automatically. This for example can also increase a battery life of a battery of the sensor apparatus, via which the electronics of the sensor apparatus are supplied. In one design variant, the wireless transmission of data comprises the transmission of a removal signal via which it is indicated that a removal of a medicament from the medicament container has been effected. Thus, a user can specifically trigger or confirm a removal signal in order to signal the proper removal of the medicament from the medicament container (and hence the intake of the medicament). In one design variant, for example, a removal signal only is generated or an automatically generated removal signal only is sent to a computer unit, after a user-side confirmation of the removal of a medicament has been detected, for example by detection of a particular sequence of movements, such as for example a “double tap”.

Consequently, the other function in particular can comprise a wireless transmission of a removal signal via which it is indicated that a removal of a medicament from a medicament container has been effected when a user moves the sensor apparatus attached to the medicament container in a particular way and this movement leads to the detection of the sequence of movements fulfilling at least one predefined detection criterion via the at least one acceleration sensor. In a development it can then be provided that a missing receipt of a removal signal triggered in this way at the computer unit, e.g. at a (gateway) server or a mobile terminal, in a predetermined time interval, leads to a generation and output of an alarm signal. The output alarm signal can be output acoustically, haptically and/or visually perceptibly, e.g. at the computer unit. So, an alarm signal is generated when a user does not properly move the medicament container at a time predetermined for taking the medicament and thereby consciously, in a manner evaluable by sensors, signals that a medicament has been removed, e.g. by carrying out a repeated striking or tapping movement with or at the sensor apparatus. In particular, a two-time tapping movement includes a so-called “double tap”.

In one design variant, the sensor apparatus is configured to automatically transmit data on a detected movement to the computer unit only in response to the presence of an alarm event stored in the sensor apparatus. Automatically forwarding data on a detected movement to the computer unit here consequently is effected only when the presence of an alarm event has been identified. The sensor apparatus here can be configured to check the presence of an alarm event with reference to the detected movement. Thus, when it is concluded for example in dependence on sensor signals provided by the at least one acceleration sensor that an alarm event does exist, a transmission of the associated data to the computer unit also is effected automatically via the sensor apparatus. A particular alarm event for example exists when a movement fulfills at least one additional alarm criterion, e.g. is identified via a time period exceeding a time threshold value and/or along an adjustment path exceeding a path threshold value and/or with an acceleration exceeding an acceleration threshold value. For example, from such a movement a critical displacement of the object provided with the sensor apparatus—here a medicament container—and hence an alarm event can be inferred, as it is assumed for example that the object has been abusively displaced for too long, too far and/or too fast.

In a possible development, the sensor apparatus is configured to store data on a detected movement, for which no alarm event has been detected, in a memory of the sensor apparatus. Consequently, when no alarm event is detected by an electronic evaluation logic of the sensor apparatus and hence for example merely an uncritical event that has led to a movement of the sensor apparatus, data on this detected movement are not wirelessly transmitted directly to the computer unit, but initially held in a memory of the sensor apparatus for a future transmission to the computer unit. A corresponding future transmission here can also be consciously triggerable on the part of a user, for example by the above-described (further) sequence of particular movements that are carried out with the sensor apparatus and are detected by its at least one acceleration sensor.

In principle, the interface of the sensor apparatus can be configured for different protocols. In one design variant, the interface is adapted and provided for a wireless data transmission via Bluetooth or Zig Bee.

Another aspect of the proposed solution relates to a sensor apparatus for attachment to an object, which comprises at least one acceleration sensor via which a movement of the object can be detected when the sensor apparatus is attached to the object, an interface for a wireless transmission of data on a detected movement to a computer unit, and an electronic connection device that is configured to wirelessly transmit data from the sensor apparatus to the computer unit. Here again, a medicament container is provided as an object. The connection device is configured, in response to a sequence of two or more movements detected via the acceleration sensor and each fulfilling at least one predefined detection criterion, to trigger a transmission of data from the sensor apparatus to the computer unit within a predetermined time period.

As already explained above, a sensor apparatus according to a further aspect can also be combined with a sensor apparatus according to the other aspects and hence in particular implement a coupling operation controllable via the acceleration sensor.

The proposed solution furthermore comprises a (monitoring) system for monitoring the movements of at least one object in the form of a medicament container, wherein the system comprises at least one proposed sensor apparatus and a computer unit with which the sensor apparatus can communicate in a wireless way.

In one design variant, for example, the computer unit is a gateway to at least one server, for example to a server of a local or remote network, in particular a cloud server. The at least one server, with which the computer unit communicates as a gateway and to which the data on a detected movement are transmitted, for example can be provided for evaluating the transmitted data. For example, in dependence on the respective object to which the sensor apparatus is attached, a corresponding server provides for an evaluation for stock management, product management and/or performance monitoring.

Another aspect of the proposed solution furthermore relates to a method for coupling a sensor apparatus to a computer unit. The sensor apparatus here is provided for attachment to an object in the form of a medicament container and, beside at least one acceleration sensor via which a movement of the object can be detected when the sensor apparatus is attached to the object, includes an interface for a wireless transmission of data on a detected movement to the computer unit. In the proposed method, coupling of the sensor apparatus to the computer unit in response to a sequence of at least two movements of the sensor apparatus is triggered for a subsequent transmission of data from the sensor apparatus to the computer unit, which movements are detected via the at least one acceleration sensor, each fulfill at least one predefined detection criterion and follow each other within a predetermined time period.

Another aspect of the proposed solution furthermore relates to a method for the transmission of data from a sensor apparatus to a computer unit. The sensor apparatus here is provided for attachment to an object in the form of a medicament container and, beside at least one acceleration sensor via which a movement of the object can be detected when the sensor apparatus is attached to the object, includes an interface for a wireless transmission of data on a detected movement to the computer unit. In the proposed method, a transmission of data from the sensor apparatus to the computer unit, can be triggered in response to a sequence of at two or more movements of the sensor apparatus, which movements are detected via the at least one acceleration sensor, each fulfill at least one predefined detection criterion and follow each other within a predetermined time period.

A design variant of a proposed method thus can be implemented in particular by a design variant of a proposed sensor apparatus. The advantages and features of design variants of a proposed sensor apparatus as explained above and below thus also apply for design variants of a proposed coupling method, and vice versa.

In accordance with the proposed solution, there is also provided a medicament container for storing at least one medicament, which includes an electronic monitoring device for monitoring the removal of the medicament from the medicament container, wherein the electronic monitoring device comprises at least one acceleration sensor. Via the at least one acceleration sensor a movement can be detected on removal of the at least one medicament, and a removal signal representative of the removal can be generated electronically. The monitoring device is configured to generate an alarm signal when no removal signal has been generated in a predefined time window by means of the at least one acceleration sensor.

Thus, according to this aspect, the proposed solution provides the use of an acceleration sensor on the medicament container, via which a movement of the medicament container and/or a movement at the medicament container can be detected in the case of a (manual) removal of the at least one medicament. When a permissible removal signal or a removal signal classified as permissible, which has been generated by means of the acceleration sensor, is not detected in a predefined time window, it is assumed that the removal of the medicament has not been effected as intended and thus, the medicament neither has been taken by the user at a predetermined time. Correspondingly, an alarm signal then is generated as a result, which for example can be used to alert the user acoustically and/or visually to take the medicament and/or to inform at least one other person, for example by means of an automatically sent voice and/or text message, about the failure to take the medicament. By using an acceleration sensor, it is possible comparatively easily and at low cost to electronically monitor the medicament container.

Here, it is assumed that with a medicament container to be handled manually, the removal of a medicament always is accompanied by a characteristic movement of the medicament container and hence an acceleration measurable by the at least one acceleration sensor or a particular sequence of possibly changing accelerations. A corresponding sequence of movements in turn leads to a (sensor) signal of the at least one acceleration sensor representative of the same, which can be assessed as a removal signal. Such a removal signal representative of a removal then can also be distinguished from other signals, which are generated for example by the at least one acceleration sensor when the medicament container is displaced otherwise, for example merely is shifted on a surface, but not lifted. By a particular design of the medicament container it can also be achieved that a storage element provided thereon, such as e.g. a compartment, only is accessible for the at least one medicament when the medicament container is manually handled in a particular way and in particular is lifted, which in turn leads to the generation of a representative removal signal during proper handling for the removal of the at least one medicament from the medicament container.

A removal signal representative of the removal can be stored in a memory of the monitoring device and hence be predetermined. For example, the removal signal can be taught-in. The removal signal here can include characteristic measured values and curves, and for example can be characteristic of an acceleration exceeding at least one acceleration threshold value and/or of an acceleration lasting for a minimum period of time along a direction of movement and/or two accelerations following each other within a time interval along mutually opposite directions of movement (which for example are representative of lifting and depositing the medicament container or a part thereof, for example a cassette of a medicament container designed as a medicament box including the compartment), so that the representative removal signal allows to infer a particular handling of the medicament container, which is typical for a proper manual removal of a medicament from the medicament container. In particular, a signal of the acceleration sensor can be relevant for the removal signal which occurs when the medicament container or a part thereof is tipped by at least 80°, in particular by at least 120°. Such tipping then for example speaks for a tipping movement carried out manually, by which a medicament is tipped onto the hand of a user or onto a surface. The representative removal signal predetermined in this way can thus also be distinguished from other signals generated by the at least one acceleration sensor, which can occur in the case of an improper use of the medicament container and/or an accidental movement of the medicament container.

Furthermore, the representative removal signal must occur within a predefined time window in order to avoid the generation of an alarm signal. When the removal signal has not occurred within the predefined time window, or a signal which has been classified as permissible and hence as representative of a removal of a medicament has not been generated, the alarm signal will be generated. In this way, a medicament container to be handled manually can also be provided with an electronic monitoring device via a comparatively inexpensive acceleration sensor.

In response to the alarm signal, an acoustically, haptically and/or visually perceptible alarm can be output at the medicament container via the monitoring device. For this purpose, the monitoring device for example can comprise a corresponding acoustic and/or optical alarm transmitter on the medicament container. Alternatively or additionally, an interface can be provided for communication with a mobile terminal, e.g. a smartphone, a smartwatch or an electronic bracelet, in order to trigger an acoustically, haptically and/or visually perceptible alarm thereon in response to the alarm signal.

Alternatively or additionally, the alarm signal can be wirelessly transmittable via the monitoring device to a computer unit spatially separated from the medicament container. In such a case, for example, the alarm signal then can additionally or exclusively lead to the fact that an acoustically and/or visually perceptible alarm is output at a computer unit spatially separated from the medicament container and/or via the computer unit further measures are triggered in response to the alarm signal, such as for example the sending of at least one voice and/or text message. The sending of a voice and/or text message in particular includes the making of an emergency call, in particular when the intake of the medicament is time-critical and possibly vitally important. The spatially separated computer unit in particular can be a mobile terminal, such as for example a smartphone, or a stationary alarm box that is coupled to the medicament container or to a plurality of medicament containers in a wireless or wire-bound way.

In principle, a proposed medicament container can form part of a monitoring system. Such a monitoring system then for example can also comprise a plurality of medicament containers, in order to enable centralized electronic monitoring of a plurality of resident-specific and/or patient-specific medicament containers in a nursing home or hospital, for example. Such a monitoring system in principle can comprise an application programming interface (API) for transmitting data to third-party software.

While in the medicament container proposed above the electronic monitoring device forms part of the medicament container, this is not absolutely necessary in accordance with the proposed solution. For example, there can also be provided a monitoring system in which at least one acceleration sensor is provided on the medicament container, but the electronic monitoring device for monitoring the removal of at least one medicament from the medicament container (including monitoring the removal of at least one medicament from a compartment of a medicament box) is at least partly spatially separated from the medicament container. The at least one acceleration sensor then for example transmits merely signals on occurring accelerations and hence movements to a spatially separated monitoring device equipped with an evaluation logic implemented on the hardware side and/or software side. Only this monitoring device, which is provided away from the medicament container, then evaluates the signals of the acceleration sensor and checks in how far a representative removal signal is present therewith. Then for example merely at least one acceleration sensor can be arranged on the medicament container, which is equipped or coupled with a transmitter for the wireless transmission of signals generated by the acceleration sensor.

The evaluation logic can be integrated for example in an alarm box coupled to the medicament container. This alarm box (as a stationary computer unit coupled to the medicament container) then is configured to receive and evaluate the signals from the at least one acceleration sensor. The evaluation here includes a classification of the extent to which a received signal (in a predefined time window) of the acceleration sensor corresponds to a removal signal representative of a removal. The necessary evaluation logic can be formed on the hardware side via a microcontroller. However, an implementation on the software side is also possible.

A software-side implementation of an evaluation logic for evaluating signals of the at least one acceleration sensor provided on the medicament box is provided in one design variant, in which the evaluation is made for example via a software application (short: app) installed in a mobile terminal. This in particular includes the fact that the monitoring device is implemented with an app on a smartphone and the at least one acceleration sensor of the medicament container transmits signals to this mobile terminal merely via a standardized interface for a wireless data transmission, such as for example WiFi, Bluetooth or ZigBee.

An at least partly spatially separated monitoring device furthermore can of course also provide the possibility that in response to a generated alarm signal an acoustically and/or visually perceptible alarm can be triggered at the medicament container itself. Alternatively or additionally, however, an acoustically and/or visually perceptible alarm can also be triggered at a monitoring device which possibly is located close to the medicament container.

In one design variant—regardless of whether the electronic monitoring device is provided on the medicament container or is spatially separated from the medicament container—the monitoring device is equipped with a gateway to at least one server and/or adapted for sending at least one voice and/or text message via a wire-bound or wireless network and/or for triggering a haptically perceptible alarm at a mobile terminal in response to the alarm signal. In the present case, network is understood to be in particular a local or non-local network. A local network for example is a PAN (“Personal Area Network”) or LAN (“Local Area Network)”). A non-local network for example comprises a WAN (“Wide Area Network”) or GAN (“Global Area Network”) and thus in particular the Internet or the telephone network. A server to be reached via a gateway for example is a server of a local or non-local network. In particular, this can be a cloud server. Via the connection to at least one server, for example, a more complex evaluation of signals provided via the at least one acceleration sensor can be effected. Alternatively or additionally, the at least one server can be used for storing and/or transmitting data on uses of the medicament container detected via the acceleration sensor. In one design variant, the server can be adapted for sending at least one voice and/or text message via a wire-bound or wireless network and/or for triggering a haptically perceptible alarm at a mobile terminal in response to the alarm signal.

Alternatively, the monitoring device on the side of the medicament container or spatially separated from the medicament container can be coupled to a (stationary or mobile) computer unit of the monitoring system, which
comprises a gateway to at least one server and/or
is adapted for sending at least one voice and/or text message via a wire-bound or wireless network and/or for triggering a haptically perceptible alarm at a mobile terminal in response to the alarm signal.

For example, the at least one server here as well can then be used for storing, evaluating and/or transmitting data on uses of the medicament container detected via the acceleration sensor.

In principle, the monitoring device—on the side of the medicament container or spatially separated from the medicament container—can be coupled to a computer unit via which the time window can be defined. For example, via a stationary alarm box configured specifically for coupling to one or more medicament containers and/or via an app of a mobile terminal it can be definable when a removal of a medicament from the medicament container has to be effected and hence a representative removal signal must be present in order to avoid the generation of an alarm.

In principle, the medicament container can be configured for example as a medicament box that comprises a carrier and at least one cassette, wherein at least one compartment provided for storing the medicament then is provided on the cassette and the cassette is releasably connected to the carrier. Depending on the design of the medicament box, the at least one acceleration sensor can be provided on the (cassette) carrier or on the cassette. In particular in a multi-part medicament box, a plurality of acceleration sensors can also be provided, whose signals are evaluated individually or in combination in order to decide on the presence of a permissible removal signal.

In one design variant it can be provided that the output of an alarm is stopped again as soon as the representative alarm signal still is detected within a time interval following the predefined time window. Then, for example, a user of the medicament container can again stop the output of the alarm when the user has detected that the medicament has not been taken at the predefined time. Thus, triggering and deactivating the alarm can easily be realized, wherein for deactivation the presence of the specific representative removal signal possibly is checked as well in order to exclude that an unspecific, slight lifting and depositing of the medicament container or of a part thereof (for example of a cassette including a compartment on a medicament box) again already leads to stopping of the alarm.

In one development it can be provided that in response to the alarm event, alarms or notices escalating step by step are output for a user of the medicament container (at the medicament container and/or at a mobile terminal of the user) in order to alert the user to the apparently missed removal of the medicament from the medicament container, in particular before a voice and/or text message is sent to third parties. For example, several (at least two) notices are output to the user with a time offset relative to each other, when no intended intake of a medicament could be detected electronically in the meantime, before a voice and/or text message is sent to third parties.

Alternatively or additionally it can be provided that stopping of the output of an alarm does not prevent that the triggering of the alarm itself is logged nevertheless in order to also inform for example family members or the nursing staff that the medicament apparently has not been taken at the predetermined time within the predefined time window.

In accordance with the proposed solution it is also possible that several time windows and in particular time windows different each day can be predefined, depending on which medication is provided for the individual user of the medicament container.

Moreover, the sending of a voice and/or text message made in response to an alarm signal cannot only be effected to an individual person or to an individual address. Rather, it is also possible that several addresses and in particular telephone numbers are stored in the monitoring device, to which a voice and/or text message is sent in response to a generated alarm signal.

By monitoring a medicament container as proposed by means of a comparatively inexpensive acceleration sensor, easy retrofitting for electronic monitoring can also be realized on commercially available, manually handleable medicament containers. The proposed solution also provides a retrofit kit for an electronic monitoring system in which at least one acceleration sensor to be attached to a medicament container and a monitoring device coupled to the at least one acceleration sensor are provided. The monitoring device then is adapted to classify an electronic signal generated by the at least one acceleration sensor as a removal signal representative of the removal of a medicament from the medicament container and to generate an alarm signal when no removal signal was generated in a predefined time window (stored in a memory of the monitoring device), and hence no removal signal classified as permissible was identified.

The proposed retrofit kit for a medicament container thus comprises in particular an acceleration sensor to be attached to the medicament container, whose generated signals can be evaluated by at least one electronic monitoring device of the retrofit kit or of an evaluation logic of the monitoring device and can be classified as to whether or not a representative removal signal hence is present. In how far an electronic signal generated by the at least one acceleration sensor then is to be classified as a removal signal representative of a removal depends on predefined parameters. For example, the signal must refer to at least one acceleration exceeding an acceleration threshold value and/or an acceleration lasting for a minimum period along a direction of movement and/or two accelerations following each other within a time interval along two mutually opposite directions of acceleration, depending on what is predetermined as characteristic for the respective medicament container for a proper manual removal of a medicament from the medicament container and for example has been taught in. As already mentioned above, this for example includes the fact that the signal of the acceleration sensor is relevant for the removal signal, which occurs on tipping of the medicament container or a part thereof by at least 80°, in particular by at least 120°.

In principle, the monitoring device of a retrofit kit can comprise an evaluation logic for the assessment of signals of the at least one acceleration sensor and for generating the alarm signal. The monitoring device can also form part of a computer unit of the retrofit kit spatially separated from the medicament container, which is provided for the (possibly wireless) reception of signals generated by the at least one acceleration sensor. Thus, the at least one acceleration sensor can specifically be coupled (“paired” in technical terms) to the monitoring device after attachment to the medicament container, in order to configure and initialize the monitoring system provided with the retrofit kit.

Alternatively, the monitoring device with its evaluation logic can likewise be provided for attachment to the medicament container. Together with the acceleration sensor, the monitoring device for example can be integrated in a sensor unit which can subsequently be attached to a medicament container. Such a local monitoring device on the side of the medicament box then for example is adapted to send alarm signals to a coupled computer unit. In other words, such a design variant then already includes an evaluation of the sensor signals provided by the at least one acceleration sensor at the medicament container and possibly also an acoustically, haptically and/or visually perceptible output of an alarm. At least in addition, however, possible alarm signals then are also each transmitted—preferably wirelessly—to a coupled computer unit, for example a stationary alarm box or a mobile terminal. Thus, the evaluation logic can also be implemented in a software application of the retrofit kit, which is to be installed on the mobile terminal.

In accordance with the proposed solution there is furthermore provided a method for providing an electronic monitoring system for a medicament container, in particular for a medicament container to be handled manually. A proposed method includes a least the following steps:
providing the medicament container intended for storing at least one medicament, and
attaching at least one acceleration sensor to the medicament container, which is coupled to an electronic monitoring device, wherein the electronic monitoring device is adapted to assess an electronic signal generated by the at least one acceleration sensor as a removal signal representative of the removal of a medicament from the medicament container and to generate an alarm signal when no removal signal was generated in a predefined time window (stored in a memory of the monitoring device).

A proposed method in particular in combination with a design variant of a proposed retrofit kit thus is suitable for retrofitting a commercially available medicament container with an electronic monitoring system that comprises at least one acceleration sensor for monitoring the removal of a medicament from the medicament container.

Although the use of at least one acceleration sensor for monitoring a medicament container also offers considerable advantages for retrofitting commercially available medicament containers at low cost, the proposed solution is of course not limited thereto. In particular, the proposed solution—as explained above and below—also includes the fact that there are provided pre-configured medicament containers equipped already with at least one acceleration sensor, and electronic monitoring devices on the side of the medicament container or spatially separated from the medicament container, which comprise a corresponding evaluation logic implemented on the hardware side and/or on the software side, can infer the presence of a representative removal signal with reference to (sensor) signals provided by the at least one acceleration sensor, which removal signal is characteristic of a handling of the medicament container on removal of a medicament from the medicament container.

In principle, an electronic monitoring device in particular can comprise at least one design variant of a sensor apparatus as explained above, so that in this respect features explained above and below in particular apply for corresponding design variants of a proposed monitoring system.

### BRIEF DESCRIPTION OF THE DRAWINGS

The attached Figures by way of example illustrate possible design variants of the proposed solution.

FIG. 1 schematically shows a system for monitoring movements of at least one object by means of a sensor apparatus to be attached to the object.

FIG. 2 shows a comparison of two different functions of the sensor apparatus, which can be triggered solely via a predefined sequence of defined movements of the sensor apparatus within a respectively defined time period.

FIG. 3 shows an exemplary overview of objects to which the sensor apparatus can be attached in principle, in order to monitor a movement of the respective object concerning the occurrence of critical alarm events as well as uncritical movement events.

FIGS. 4A-4B show exemplary representations of a medicament box to which a sensor apparatus is attached according to the proposed solution.

FIGS. 5A-5C show exemplary representations of further medicament containers to which a sensor apparatus is attached according to the proposed solution.

FIG. 6 shows a design variant of a proposed monitoring system, in which at least one acceleration sensor is provided on a medicament box in order to infer a handling of the medicament box for removing at least one medicament within a predefined time window via sensor signals generated by the at least one acceleration sensor.

FIG. 7 shows another design variant for an alternatively designed medicament box.

### DETAILED DESCRIPTION

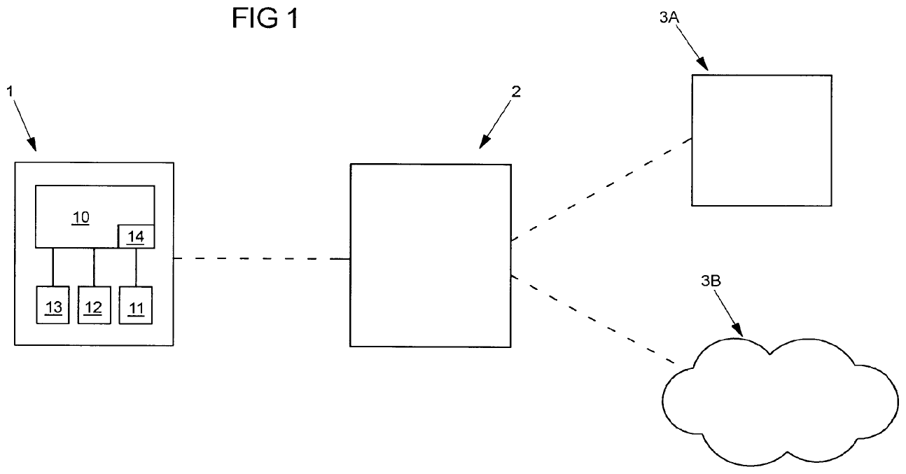

FIG. 1 schematically shows a system for monitoring the movement of an object to which a sensor apparatus 1 is attached. Beside a connection device 10 comprising an electronic evaluation logic (which can form part of an electronic monitoring device), the sensor apparatus 1 comprises at least one inertial sensor 11 (or at least one acceleration sensor which forms part of the inertial sensor 11), an interface 12 for a wireless data transmission, a memory 13 and a battery 14. Via the interface 12, the sensor apparatus 1 can wirelessly communicate with a computer unit 2, for example a gateway, and transmit data collected and/or generated by means of the inertial sensor 11 and the connection device 10 to the computer unit 2. When the computer unit 2 acts as a gateway, the data can be transmitted for example to a local server 3A or a cloud server 3B in order to be evaluated and further processed there.

In accordance with the proposed solution it is provided that the connection device 10 with its electronic evaluation logic of the sensor apparatus 1 (e.g. implemented via at least one microcontroller) is configured to trigger particular functions of the sensor apparatus 1 in response to a sequence of at least two movements detected and predefined via the inertial sensor 11 within a predetermined time period. This includes for example coupling of the sensor apparatus 1 to the computer unit 2 and hence the first-time establishment of a data connection between the sensor apparatus 1 and the computer unit 2 for a subsequent wireless transmission of data by means of the interface 12 of the sensor apparatus 1. When the computer unit 2 and the sensor apparatus 1 for example communicate in a wireless way via Bluetooth or ZigBee, a pairing of the sensor apparatus 1 with the computer unit 2 can be triggered at the sensor apparatus 1 by a sequence of movements of the sensor apparatus 1, without this requiring a further actuation of a separate operating element at the sensor apparatus 1.

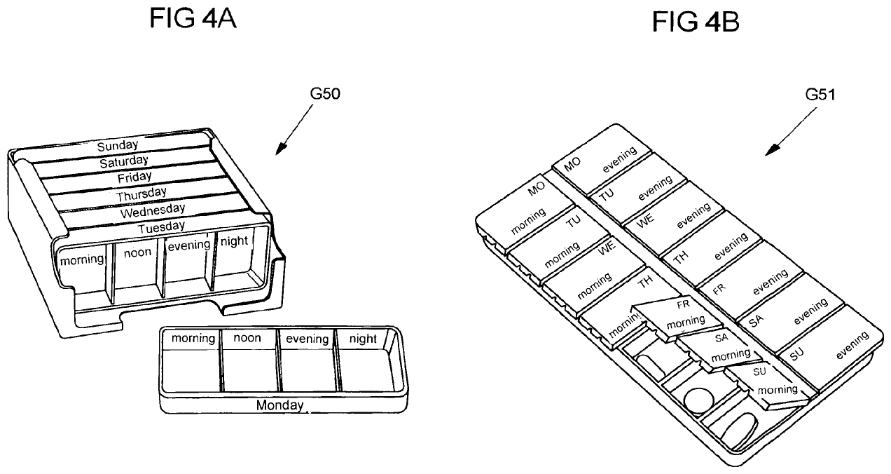

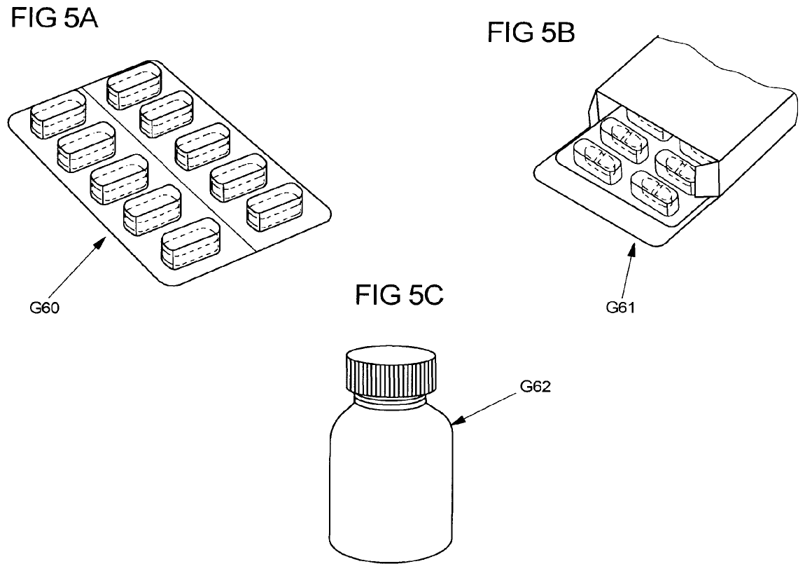

For example, the connection device 10 is configured such that by a two-time striking or tapping movement (in particular e.g. in the manner of a “double tap”), which is carried out with the sensor apparatus within a predefined time window of for example 5 seconds, a coupling operation, in particular a pairing process, is automatically started. When this coupling operation is successfully completed, data can be transmitted from the sensor apparatus 1 to the computer unit 2, which data characterize a movement subsequently carried out with the sensor apparatus 1. Since a coupling operation, in particular a pairing process, can be triggered without an operating element at the sensor apparatus 1, the coupling operation also is possible after a hidden attachment of the sensor apparatus 1 to the object to be monitored. The object to be monitored for example is a medicament box G50 or G51, as it is shown by way of example in FIGS. 4A and 4B, or another medicament container. Further exemplary medicament containers for example include a pill blister G60 of FIG. 5A, a medicament package G61 of FIG. 5B or a medicament bottle G62 of FIG. 5C, which for example contains a liquid or powered medicament.

Corresponding (event) data on at least one of the detected movements of the sensor apparatus 1 can comprise for example a starting time and/or end time and/or duration of a detected movement, type and/or direction of the movement, position data on at least one position reached with the detected movement and/or a date and/or time indication. With reference to the generated data, it can thus be evaluated at the computer unit 2 and/or at one of the servers 3A, 3B in how far an object provided with the sensor apparatus 1 was moved. After a coupling operation was triggered by identifying a sequence of defined (atypical) movements such as for example a sequence of striking or tapping movements, within 5 seconds, and is successfully completed without having to actuate an operating element such as a tip switch at the sensor apparatus 1, it consequently can be monitored via the sensor apparatus 1 for example whether an object is moved or was moved at certain times of the day, as intended, and/or whether an object was abusively displaced, in particular is or was removed from a particular building.

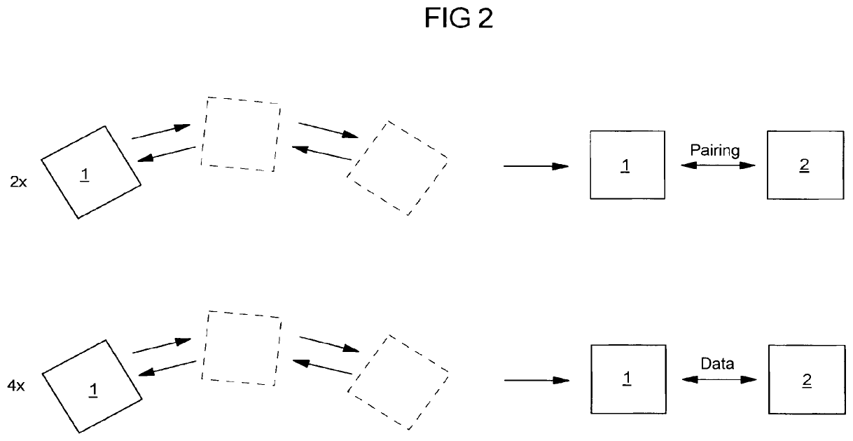

Corresponding to the representation of FIG. 2, a proposed sensor apparatus 1 also can provide that in response to a subsequently electronically detected (by means of the evaluation logic of the connection device 10) sequence of predefined movements another function can also be triggered at the sensor apparatus 1 within a predetermined time window. For example, when after the successful coupling of the sensor apparatus 1 and the computer unit 2, the sensor apparatus 1 repeatedly accelerates abruptly four times within 5 seconds along a predetermined spatial direction, or twice carries out a double tap movement, this will trigger a one-time transmission of stored data on previously made and detected movements of the sensor apparatus 1 to the computer unit 2. Thus, an evaluation logic of the connection device 10 is configured to distinguish between critical and uncritical movement events. When a detected movement is classified as critical and hence for example as indicating an abusive behavior, an alarm event exists, which triggers an automatic immediate transmission of the data belonging to this movement to the computer unit 2. On the other hand, when a movement is classified as uncritical and hence as not resulting from an alarm event, data on the detected movement are merely stored in the memory 13 of the sensor apparatus 1. These data are transmitted to the computer unit 2 only when needed in the case of an operating event triggered on the part of a user—here by the four-time acceleration of the sensor apparatus 1 as outlined in FIG. 2. In this way, a service life of the battery 14 of the sensor apparatus 1 can be maximized, as not all detected movements and hence movement events are transmitted to the computer unit 2.

Rather, what is automatically transmitted to the computer unit 2 merely are data on those movements which fulfill an additional alarm criterion and hence also indicate the presence of an alarm event. Stored data on the detected movements, which are not classified as belonging to an alarm event, on the other hand only are transmitted to the computer unit 2 specifically in response to an interaction on the part of the user.

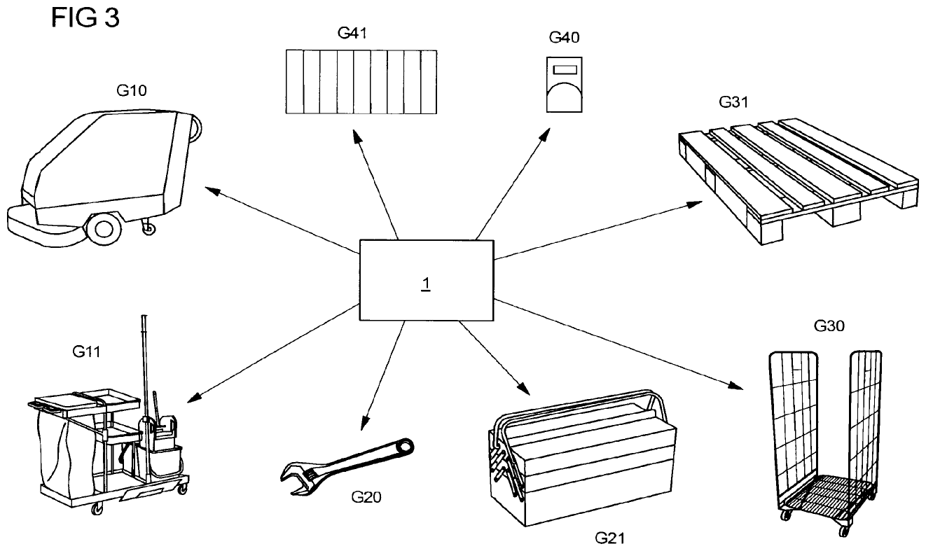

FIG. 3 by way of example illustrates different objects G10 to G41, to which the sensor apparatus 1 can likewise be attached.

For example, the attachment to a cleaning device G10 or to a cleaning utensil or a carrier G11 receiving such cleaning utensil is possible in order to electronically identify a scenario of when and where cleaning with the respective cleaning device G10 or cleaning utensil has taken place.

When the sensor apparatus 1 is attached to a tool G20 or a tool box G21, it can be detected whether a tool is stolen from a production facility, effectively and in a battery-saving way by means of the sensor apparatus 1.

The attachment of the sensor apparatus 1 to a grid trolley G30 or another goods carrier, such as a pallet G31, for example, allows monitoring in the course of warehousing. Then, for example, a removal or movement of goods can be detected. Furthermore, the time of the corresponding movement can be compared with the stored time for a picking order, in order to determine whether an authorized removal of goods does exist.

When attached to an individual item G40 or a goods package G41, it is also possible to electronically monitor whether a removal of goods and thus their movement is permissible, or whether for example there is a movement which is assessed as critical and hence as triggering an alarm event.

However, the focus of the present application in particular is a sensor apparatus that is adapted and provided for attachment to a medicament container, and a medicament container comprising a proposed sensor apparatus. In principle, such a sensor apparatus can also form part of an electronic monitoring system for which exemplary design variants are explained below.

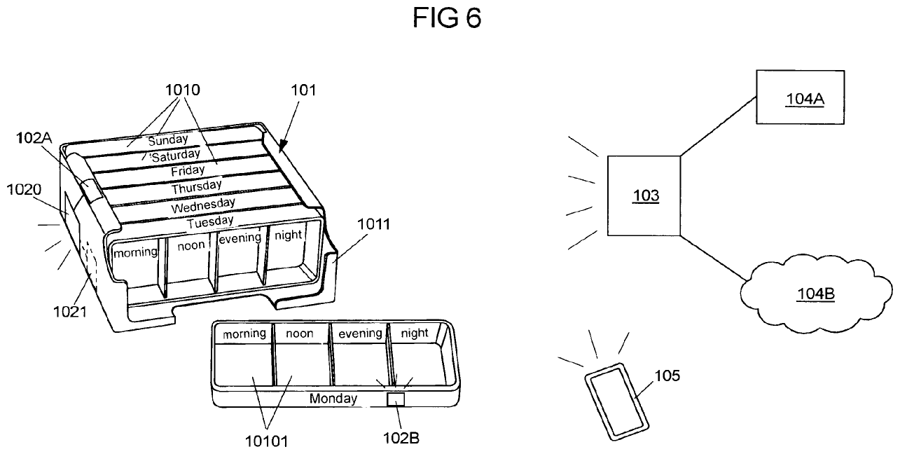

In a monitoring system of FIG. 6, a medicament box 101 by way of example is provided as a medicament container, which on a carrier in the form of a supporting frame 1011 provides a plurality of cassettes 1010 each having at least one compartment 10101 for medicaments. In the present case, seven cassettes are provided—one cassette for each day of the week.

Furthermore, each cassette 101 in the present case by way of example includes four compartments 10101, which then for example can contain medicaments for different time windows, for example morning, noon, evening and night.

On the supporting frame 1011 of the medicament box 101 an acceleration sensor 102A is provided, which is coupled to an electronic evaluation unit 1020 of an electronic monitoring device. The electronic evaluation unit 1020 likewise is provided on the supporting frame 1011. The electronic evaluation unit, for example in the form of at least one microcontroller, implements an evaluation logic on the hardware side in order to evaluate with reference to sensor signals generated by the acceleration sensor 102A during a movement of the supporting frame 1011 whether the supporting frame 1011 is handled in such a way that it can be concluded that a cassette 1010 is removed from the supporting frames 1011. Thus, handling of the supporting frame 1011 on removal of a cassette 1010 for removing a medicament involves a particular sequence of movements which can be electronically evaluated and hence be identified with reference to sensor signals supplied by the acceleration sensor 102A. Alternatively or additionally, a (possibly additional) acceleration sensor 102B can be provided on each cassette 1010 of the medicament box 101. Using sensor signals of the cassette-side acceleration sensor 102B, which are provided as an alternative to the sensor signals of the support frame-side acceleration sensor 102A or in addition to the sensor signals of the support frame-side acceleration sensor 102A, a proper use of the medicament box 101 for removing a medicament from one of the compartments 10101 can likewise and possibly even more specifically be inferred via the electronic evaluation unit 1020. For example, with reference to a signal of the acceleration sensor 102B it is inferred whether the respective cassette 1010 is tipped by at least 80°, in particular by at least 120°, in order to tip a medicament out of a compartment 10101.

The electronic evaluation unit 1020 possibly also includes a timing device or timer in order to assess whether a sensor signal is supplied by at least one of the acceleration sensors 102A, 102B within a predefined time window, which is to be classified as a removal signal characteristic of a removal of a medicament from a compartment 10101. When no sensor signal representative of the removal of a medicament and hence classified as permissible is received or no corresponding combination of sensor signals of the two acceleration sensors 102A, 102B is received within the predefined time window, an alarm signal is generated by the electronic evaluation unit 1020 in order to alert to a missed removal of a medicament.

Via the alarm signal of the electronic evaluation unit 1020 an alarm transmitter 1021 for example can be actuated at the medicament box 101. By way of example, the alarm transmitter 1021 is arranged on the supporting frame 1011. Via the alarm transmitter 1021, for example an alarm that is acoustically and visually perceptible for a human user can be output in order to alert to the missed removal of a medicament.

In addition, an alarm signal of the electronic evaluation unit 1020 can be transmitted to a computer unit in the form of a stationary alarm box 103, which is coupled to the electronic evaluation unit 1020. This alarm box 103 forms part of the monitoring system, which here is not only provided for outputting an acoustically and visually perceptible alarm at the medicament box 101, but also specifically fulfills further (alarm) functions. The alarm box 103 for example can serve as a gateway to a local server 104A or to a cloud server 104B. The alarm box 103 therefor is coupled to the corresponding server 104A, 104B via a local or non-local network.

Alternatively or additionally, the alarm box 103 can be coupled to a plurality of medicament boxes 101 which each comprise an electronic monitoring device with an electronic evaluation unit 1020 for generating alarm signals which then can wirelessly communicate with the alarm box 103 via an integrated transmitter.

For example, the alarm box 103 also performs the sending of voice and/or text messages via a wire-bound or wireless network and/or for triggering a haptically perceptible alarm at a mobile terminal in response to an alarm signal of a medicament box 101, for example in order to alert family members and/or the nursing staff to a missed intake of a medicament.

In an alternative design variant, the electronic evaluation unit 1020 for evaluating the sensor signals generated by an acceleration sensor 102A, 102B is not provided at the medicament box 101, but implemented in the alarm box 103. Signals of an acceleration sensor 102A, 102B thus are sent to the alarm box 103 (and/or another computer unit), which compares these signals with the stored removal signal—or at least with the parameters stored as relevant therefor—and then generates an alarm signal possibly by taking account of the at least one stored time window.

Alternatively or additionally, the electronic monitoring device of a medicament box 101 can also be coupled to a mobile terminal 105. Such a mobile terminal 105 then for example is a smartphone or a smartwatch on which a software application (short: app) is installed, which can receive an alarm signal from the medicament box-side monitoring device and/or from the alarm box 103. In this way, a local or remote user can also be alerted to a possibly missed removal of a medicament by means of the mobile terminal 105, which was identified by means of an acceleration sensor 102A or 102B or by means of a plurality of acceleration sensors 102A, 102B. Alternatively or additionally, the electronic monitoring device can be coupled to a mobile terminal in the form of an electronic bracelet, so that an acoustically, haptically and/or visually perceptible alarm is triggered thereon in response to the alarm signal.

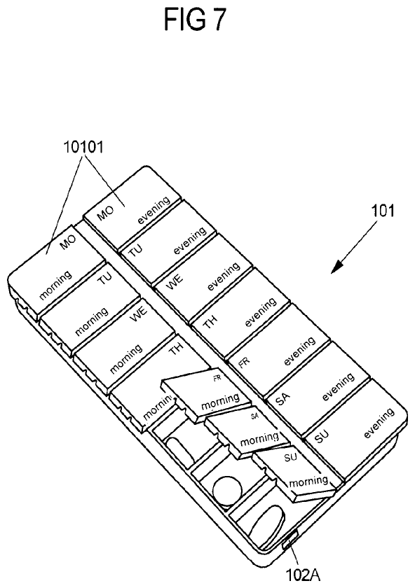

FIG. 7 shows an alternative design variant of a cassetteless medicament box 101, which can likewise be used in accordance with the proposed solution. The medicament box 101 of FIG. 7 includes a plurality of fixed medicament compartments 1010 to be individually closed by a lid, wherein each compartment 10101 typically is provided for taking the medicament held there at a particular day and/or at a particular time of the day. An acceleration sensor 102B likewise is provided at the medicament box 101 of FIG. 7, which is coupled e.g. to the alarm box 101 of FIG. 6 in order to be able to wirelessly transmit signals to this alarm box 103. By means of the acceleration sensor 102B it can be detected in particular whether the medicament box 101 was manually tipped within a predetermined time window, which allows to conclude that a medicament was tipped out of an (open) compartment 10101.

By means of one or more acceleration sensors 102A, 102B on a medicament box 101, it is not only comparatively easily possible to equip a medicament box 101 with an electronic monitoring system. Rather, the configuration of the monitoring system can also be realized comparatively easily. For example, the acceleration sensors 102A, 102B—or inertial sensors comprising the same—can be automatically paired with the alarm box 103 and/or the mobile terminal 105. By means of an app installed on the mobile terminal 105 or by means of a software of the alarm box 103, the medicament box 1 can be assigned to a particular user or patient, respectively. A medicament intake schedule with different predefined time windows can also be configured on the software side. Correspondingly, different alarm options can then also be predetermined. For example, it can then be predefined whether an alarm is to be generated via the alarm transmitter 1021 so as to be only acoustically and/or visually perceptible, to which addresses or phone numbers text and/or voice messages are sent in the case of an alarm, and whether the generation of an alarm signal possibly is to be omitted (for example because the assigned user does not use the medicament box 1 for a particular time due to vacation).

Configurations can also be made via the acceleration sensor 102A or 102B. For example, the acceleration sensor 102A, 102B can be adapted such that a particular function is triggered within a predetermined time period by two movements detected via the acceleration sensor 102A, 102B and each fulfilling at least one predefined detection criterion, as this for example has already been explained above in connection with a function of coupling and/or data transmission. For example, a two-time striking or tapping movement (in particular for example in the manner of a so-called “double tap”), which is carried out by means of the acceleration sensor 102A or 102B within a predefined configuration time interval, of for example 5 seconds, can lead to the generation of a reset signal or removal signal. Via a reset signal, for example the coupling to the alarm box 3 and/or the mobile terminal 5 then is reset again, and a new pairing becomes possible. With a removal signal it is signaled and hence possibly specifically confirmed on the part of the user that a medicament has been removed from the medicament box 101.

After the configuration of the monitoring system, the acceleration sensor 102A or 102B can not only be used for the (automatic) detection of a representative removal signal. The respective acceleration sensor 102A or 102B rather can also be used to again deactivate an alarm once activated, for example by carrying out a particular movement with the supporting frame 1011 including the acceleration sensor 102A or with the cassette 1010 carrying the acceleration sensor 102B, such as a “double tap”.

Analogously, by carrying out a particular movement it can be confirmed on the part of the user that a medicament has been removed from the medicament box 101. In one design variant, an automatically generated removal signal is sent to the alarm box 103 and/or the terminal 105 only after a user-side confirmation of the removal of a medicament has additionally been detected, for example by detection of a particular movement with the supporting frame 1011 including the acceleration sensor 102A or the cassette 1010 carrying the acceleration sensor 102B, such as a “double tap”. Alternatively, a permissible removal of a medicament from the medicament box 101 is detected only by carrying out a particular movement with the supporting frame 1011 including the acceleration sensor 102A or the cassette 1010 carrying the acceleration sensor 102B, such as a “double tap”, and as a result of a detection of such a movement, like a “double tap”, the generation of a removal signal is triggered.

The output of an acoustically, haptically and/or visually perceptible alarm furthermore can also be effected with a time delay, in order to send a voice and/or text message by means of the alarm box 103 and/or the mobile terminal 105 so that an electronically detected removal of a medicament after triggering the alarm still is sufficient to possibly avoid the sending of a voice and/or text message to family members and/or the nursing staff. For example, the sending of a voice and/or text message only can be effected automatically when, after generating an alarm signal within a deactivation time interval of e.g. 3, 5 or 7 minutes, a removal signal classified as permissible is still not identified or for example no particular deactivation movement is carried out with the medicament box, such as for example a “double tap”.

In one design variant, the monitoring system furthermore can be adapted to trigger additional alarm signals which for example alert to a low battery state of a battery provided for an acceleration sensor 102A or 102B and/or for the electronic evaluation unit 1020 and/or to a phase of muting alarms to be output, which is classified as atypically long (for example a phase of several days).

Alternatively or additionally, the monitoring system comprising an acceleration sensor 102A or 102B can also be adapted to classify sensor signals provided by an acceleration sensor 102A or 102B not only as a removal signal or as inadmissible, but also identify at least one further signal pattern in order to trigger an additional function. For example, via a specific movement of the medicament box 101, in particular a two-time striking or tapping movement, an emergency call can specifically be triggered, and hence the sending of a voice and/or text message can be triggered in a user-controlled way independently of the removal of a medicament at a predefined time window.

Via an app on the mobile terminal 105 and/or on the alarm box 103 in one design variant all alarms furthermore can be logged and hence be stored in a readable way, in particular independently of whether a removal of a medicament possibly has yet been detected and/or the alarm has yet been deactivated.

The functions and advantages explained with FIGS. 6 and 7 of course are not dependent on the use of a medicament box 101. Rather, these functions and advantages can also be achieved when using another medicament container, such as e.g. a pill blister G60 of FIG. 5A, a medicament package G61 of FIG. 5B or a medicament bottle G62 of FIG. 5C.

In principle, the proposed solution in particular can include the use of at least one acceleration sensor, which has already been attached to an arbitrary medicament container (such as a pill box, pill bottle, pill carton, pill blister, bottle for liquid medicaments or powder) on the part of the manufacturer or subsequently can be attached to a medicament container, and via which a usual movement when using the medicament container is identified, as a result of which a removal signal is transmitted to a smartphone and/or a gateway PC (for forwarding to the cloud). In an alternative or additional design variant, the at least one acceleration sensor is used to identify a predetermined sequence of movements or a particular sequence of movements, e.g. a double tap of the medicament user. This sequence of movements can then for example directly trigger a data transmission to a smartphone or a gateway PC, which is interpreted as a confirmation that a medicament from the medicament container has been taken as intended.

### LIST OF REFERENCE NUMERALS

1 sensor apparatus
10 connection device
11 inertial sensor
12 interface
13 memory
14 battery (energy accumulator)
2 computer unit
3A local server
3B cloud server
101 medicament box
1010 cassette
10101 compartment
1011 supporting frame (carrier)
102A, 102B acceleration sensor
1020 electronic evaluation unit/transmitter
1021 alarm transmitter
103 alarm box
104A local server
104B cloud server
105 mobile terminal
G10-G41 object
G50, G51, G60, medicament box (medicament container)
G61, G62
G60 pill blister (medicament container)
G61 medicament package (medicament container)
G62 medicament bottle (medicament container)

1. A sensor apparatus for attachment to a medicament container, at least comprising
at least one acceleration sensor, via which a movement of the medicament container can be detected when the sensor apparatus is attached to the medicament container,
an interface for a wireless transmission of data on a detected movement to a computer unit, and
an electronic connection device, which is configured to wirelessly transmit data from the sensor apparatus to the computer unit,
wherein the connection device is configured
in response to a sequence of at least two movements detected via the acceleration sensor and fulfilling at least one predefined detection criterion, at least one of a) to trigger a transmission of data from the sensor apparatus to the computer unit and b) to trigger coupling of the sensor apparatus to the computer unit, within a predetermined time period.

2. The sensor apparatus according to claim 1, wherein the connection device is configured to trigger the coupling of the sensor apparatus to the computer unit when a sequence of at least two successive movements has been detected, during each of which an acceleration of the sensor apparatus occurs, which exceeds an acceleration setpoint value stored as a detection criterion.

3. The sensor apparatus according to claim 1, wherein the at least two successive movements each comprise an acceleration of the sensor apparatus out of an idle state and are effected along a matching spatial direction.

4. The sensor apparatus according to claim 1, wherein the connection device is configured, in response to a sequence of two or more predefined movements of the sensor apparatus detected via the acceleration sensor, to trigger a function that at least one of is different from coupling of the sensor apparatus to the computer unit and comprises a wireless transmission of data to the computer unit.

5. The sensor apparatus according to claim 4, wherein the connection device is configured to trigger the other function only after successfully coupling the sensor apparatus to the computer unit in response to the sequence of two or more predefined movements of the sensor apparatus.

6. The sensor apparatus according to claim 4, wherein the time period in which the at least two movements for triggering the coupling of the sensor apparatus to the computer unit must be effected is different from the time period in which the two or more predefined movements of the sensor apparatus must occur in order to trigger the other function.

7. The sensor apparatus according to claim 4, wherein the number of the at least two successive movements, which for triggering the coupling of the sensor apparatus to the computer unit must each fulfill a predefined detection criterion, is different from a number of two or more successive movements which must occur in order to trigger the other function.

8. The sensor apparatus according to claim 4, wherein the other function comprises a wireless transmission of data on at least one detected movement of the sensor apparatus to the computer unit, in particular the transmission of a removal signal via which it is indicated that a removal of a medicament from the medicament container has been effected.

9. The sensor apparatus according to claim 8, the other function comprises a wireless transmission of a removal signal via which it is indicated that a removal of a medicament from the medicament container has been effected by a user moving the sensor apparatus attached to the medicament container in a particular way which leads to the detection of the sequence of movements fulfilling a predefined detection criterion via the acceleration sensor.

10. The sensor apparatus according to claim 9, wherein the connection device is adapted to generate an alarm signal when no removal signal has been generated in a predefined time window, or the computer unit is configured to generate an alarm signal when no removal signal has been received by the sensor apparatus in a predefined time window.

11. The sensor apparatus according to claim 1, wherein the sequence of at least two movements detected via the acceleration sensor and fulfilling at least one predefined detection criterion comprises an at least two-time striking or tapping movement with or at the sensor apparatus, in particular a “double tap”.

12. The sensor apparatus according to claim 1, wherein the sensor apparatus is configured to automatically transmit data on a detected movement to the computer unit only in response to the presence of an alarm event stored in the sensor apparatus.

13. (canceled)

14. The sensor apparatus according to claim 11, wherein the sensor apparatus is configured to store data on a detected movement, for which no alarm event was detected, in a memory of the sensor apparatus.

15. (canceled)

16. The sensor apparatus according to claim 1, wherein the sensor apparatus being a part of a monitoring system, in which the sensor apparatus communicates in a wireless way with a computer unit.

17. (canceled)

18. The sensor apparatus according to claim 1, wherein the sensor apparatus being attached to a medicament container.

19. A method for transmitting data from a sensor apparatus to a computer unit, wherein
the sensor apparatus is provided for attachment to a medicament container and includes:
at least one acceleration sensor via which a movement of the medicament container can be detected when the sensor apparatus is attached to the medicament container, and
an interface for a wireless transmission of data on a detected movement to a computer unit;
a transmission of data from the sensor apparatus to the computer unit is triggered in response to a sequence of at two or more movements of the sensor apparatus, which movements are detected via the at least one acceleration sensor, fulfill at least one predefined detection criterion and follow each other within a predetermined time period.

20.-24. (canceled)

25. A monitoring system, comprising at least one medicament container for storing at least one medicament and an electronic monitoring device for monitoring the removal of the at least one medicament from the at least one medicament container,
wherein the medicament container comprises at least one acceleration sensor via which a movement can be detected on removal of the at least one medicament, and a removal signal representative of the removal can be generated electronically, and the monitoring device is configured at least one of
in response to a sequence of at least two movements detected via the acceleration sensor and fulfilling at least one predefined detection criterion, at least one of a) to trigger a transmission of data from the sensor apparatus to the computer unit and b) to trigger coupling of the sensor apparatus to the computer unit, within a predetermined time period, and
to generate an alarm signal when no removal signal has been generated in a predefined time window.

26. (canceled)

27. The monitoring system according to claim 25, wherein the monitoring device at least one of
comprises a gateway to at least one server and
is adapted at least one of
for sending at least one of at least one voice message and at least one text message via a wire-bound or wireless network and
for triggering a haptically perceptible alarm at a terminal in response to the alarm signal.

28.-31. (canceled)

32. The monitoring system according to claim 25, wherein the medicament container comprises a carrier and at least one cassette, wherein at least one compartment for a medicament to be removed is provided at the cassette, and the cassette is releasably connected to the carrier.

33.-34. (canceled)

35. The sensor apparatus according to claim 1, wherein the sensor apparatus being configured as a retrofit kit for an electronic monitoring system in which a removal of the at least one medicament from the medicament container is electronically monitored.

36.-40. (canceled)
